(19) United States
(12) Patent Application Publication
(10) Pub. No.: US 2009/0318788 A1
(43) Pub. Date: Dec. 24, 2009

(54) DETECTION OF CANCER MARKERS

(76) Inventors: Kalle LEVON, Brooklyn, NY (US); Miriam RAFAILOVICH, Stony Brook, NY (US); Basil RIGAS, Old Field, NY (US); Yantian Wang, Port Jefferson Station, NY (US)

Correspondence Address: FROMMER LAWRENCE & HAUG, 745 FIFTH AVENUE- 10TH FL., NEW YORK, NY, 10151, US

(21) Appl. No.: 12/109,836

(22) Filed: Apr. 25, 2008

Related U.S. Application Data
(60) Provisional application No. 60/926,500, filed on Apr. 27, 2007.

(51) Int. Cl. A61B 5/1468

(52) U.S. Cl. 600/345

(57) ABSTRACT

The present invention relates to combining surface molecular imprinting (SMI) with the production of self-assembled monolayers (SAM) of hydroxyl alkanethiolate molecules on gold coated chip surfaces. In this technique, the sensing element is placed on the transducer and the whole assembly can then be miniaturized and integrated into a smart chip. These sensors can detect, nanomolar quantities of complex biomolecules.

### INCORPORATION BY REFERENCE

This application claims priority to U.S. provisional patent application Ser. No. 60/926,500 filed Apr. 27, 2007.

Reference is made to U.S. patent application Ser. No. 10/242,590 filed Sep. 12, 2002 which published as U.S. patent publication no. US 2004-0058380 A1 on Mar. 25, 2004 and international patent application serial no. PCT/US03/28518 filed Sep. 11, 2003 which published as international patent publication no. WO 2005/03716 on Jan. 13, 2005.

The foregoing applications, and all documents cited therein or during their prosecution and all documents cited or referenced herein, together with any manufacturer's instructions, descriptions, product specifications, and product sheets for any products mentioned herein, are hereby incorporated herein by reference, and may be employed in the practice of the invention.

### FIELD OF THE INVENTION

The present invention concerns new concepts in sensing, particularly integrating molecular recognition processes and sensor transduction, and their application to detecting biological molecules.

### BACKGROUND OF THE INVENTION

High sensitivity detection of biological molecules and microbes has always been the topic of much interest since it has numerous clinical applications to early identification of cancer and other diseases. More recently, renewed interest has been sparked by concerns regarding the proliferation of biological warfare agents. In the latter case, it is also advantageous that the sensing technique provide a rapid response while at the same time be portable, robust, easy to operate, and require very small amounts of sample.

The process of molecular imprinting (MI) first suggested by Linus Pauling as an explanation for antibody formation [Pauling L. J. Am. Chem. Soc. 1940, 62, 2643], has in the last few years proven to be an effective method for recognition and detection of a wide range of chemical substances [Molecularly Imprinted Polymers, Man-Made Mimics of Antibodies and Their Application in Analytical Chemistry; Sellergren, B., Ed.; Elsevier: New York, 2001, Molecular and Ionic Recognition with Imprinted Polymers, Bartsch R. A., Maeda M., Eds.; American Chemical Society: Washington D.C., 1998].

Currently, one technique for biological detection is based on immunosensing using antibodies. Even though this technique is sensitive, the generation of specific antibodies can be time consuming and expensive. Furthermore, since physiological conditions are needed, even small deviations can impair the function of the antibodies.

Molecular imprinting using artificial materials, on the other hand, is more robust. Only a small set of molecules is required for imprinting a wide variety of antigens. These molecules do not require special physical or chemical conditions and are stable over a broad range of temperatures. The technique was further refined by Zhou et al. who demonstrated that imprinting can also be accomplished using functionalized tri-chlorosilanes which self assemble into a well ordered surface crystalline monolayer rather than functional monomers which are polymerized around the template [Zhou Y., Yu B., Shiu E., Levon K., Anal. Chem. 2004, 76, 2689]. While this approach had the advantage that the detector and transducer were integrated, one obstacle to the production of an actual biosensor was the immiscibility of the silanes in physiological solutions and the belief that only molecules comparable in size to the thickness of the self-assembled monolayer (SAM), or less than 2 nm, could be templated. This limited detection to small non-polar molecules which were mainly byproducts of other biological reactions.

There remains a need for high sensitivity detection of biological molecules and microbes wherein the sensing technique provides a rapid response while at the same time be portable, robust, easy to operate, and require small amounts of sample.

Citation or identification herein of any document is not an admission that such document is available as prior art to the present invention.

### SUMMARY OF THE INVENTION

Limitations to molecular imprinting could be removed if templating is accomplished with hydroxyl functionalized thiol hydrocarbon chains directly assembled on a gold (Au) coated electrode. The directional polar bonding of the template with the hydroxyl groups provides a further degree of selectivity and only a small portion of the key needs to be recognized by the “lock”. A chip may be designed which can recognize large complex molecules, such as proteins under physiological conditions and in concentrations as low as several nano-grams per milliliter.

The present invention relates to a surface-molecularly imprinted sensor for detecting target biological molecules. The sensor may comprise a gold plated electrode and hydroxyl functionalized thio hydrocarbon chains directly assembled on the gold plated electrode, wherein the polymer monolayer or film may be imprinted with cavities for detecting the target biological molecules. In an advantageous embodiment, the cavities are complementary to the size, geometry, and functionality of the target biological molecules. Preferably, the cavities are made by template molecules, more advantageously, target biological molecules, such as, but not limited to, carcinoembryonic antigen (CEA), cathepsin-D, poliovirus or amylase.

The present invention also encompasses methods for detecting target biological molecules using a surface-molecularly imprinted sensor. The method may comprise providing a solution containing target biological molecules and any of the sensors contemplated by the present invention, choosing a detection method, and recognizing target biological molecules based on the detection method's output. Advantageously, the detection method is potentiometry and the detection method's output may be the potential response of the solution. In a particularly advantageous embodiment, the sensor may be implanted in vivo and the detection method's output may be transmitted remotely to a detector.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIGS. 1A-1C depict a scheme of sending principle with surface molecular imprinting;

FIG. 1A depicts the cavities having the best affinity to the templating molecules;

FIG. 1B depicts the side view of the capturing geometry of the target molecules that is far larger than the SAM thickness;

FIG. 1C depicts the detailed mechanism of how the SAM captures the target molecules;

FIG. 2A depicts a CEA sensor response to CEA in PBS as compared with a non-imprinted control electrode;

FIG. 2B depicts a sensor response to medium of cell at different times;

FIG. 2C depicts a sensor response to medium containing different number of cells;

FIG. 3A depicts a cathepsin-D sensor and non-imprinted electrode response to Cath-D and

FIG. 3B depicts a poliovirus sensor response to poliovirus (), hemoglobin (◯) and non-imprinted electrode response to poliovirus (+).

FIG. 4 depicts an amylase sensor response to P-amylase.

FIG. 5 depicts a poliovirus sensor response to poliovirus () and adenovirus (◯) and a non-imprinted electrode tested with poliovirus (*).

### DETAILED DESCRIPTION

The method of the present invention is applied to the detection of a cancer marker, advantageously colorectal cancer markers, more advantageously carcinoembryonic antigen (CEA). A working device can be constructed which can be used to detect CEA generated from living cancer tissue. Hence this method may some day lead to a new generation of sensors, which when coupled to existing wireless microtechnology, can be implanted post surgically to detect recurrence of cancer at its earliest stages.

The method of the invention also is applied to detect another cancer marker, cathepsin-D, poliovirus and amylase which proved to be effective. Furthermore, by placing the sensors inside living cell culture media, the detector can sense markers being produced by cancerous tissue, in vitro, without other serum proteins affecting the sensitivity. Hence this technology can also be potentially implanted in vivo and monitored externally.

Fabricating surface-imprinted sensors involves (i) co adsorbing polymer and template molecules on the sensor's surface and (ii) removing the template molecules from the sensor's surface.

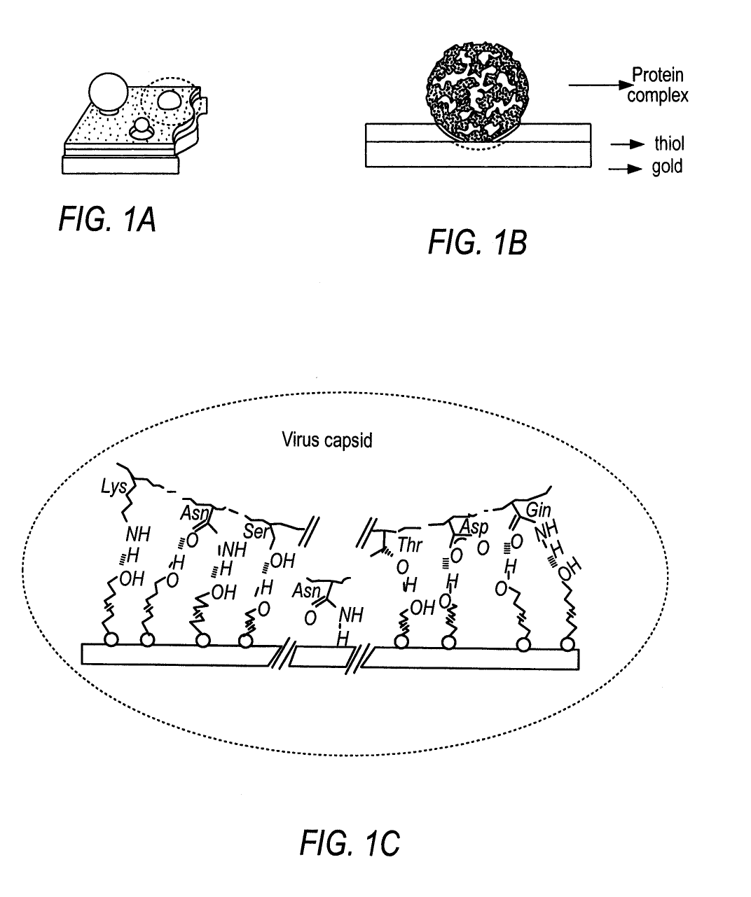

The basic principals of the surface molecular imprinting (SMI) technique are illustrated in FIG. 1A. Alkanethiol molecules with hydroxyl end groups and target biological molecules or microbes may be co-absorbed onto a substrate from the solution.

Thiol molecules chemically attached to the substrate through the sulfur-metal bond and self-assembled into stable crystalline monolayer, while the target molecules (template) are physically incorporated among the thiol monolayer.

Upon repetitive rinsing with water and phosphate buffered saline (PBS) solution, the adsorbed biomolecules are removed from the surface, leaving behind cavities which are complementary in size, shape and chemistry only with the template molecules. Hence, the sensing electrode is expected to have higher affinity to the template molecules than to other guest molecules.

The thiol molecule is an alkane thiol, advantageously a fatty alcohol thiol, more advantageously a mercapto fatty alcohol thiol, even more advantageously a 11-mercapto-1-undecanol thiol. In another embodiment, the thiol molecule may be 1-Adamantanethiol, 11-Amino-1-undecanethiol hydrochloride, Biphenyl-4,4-dithiol, Butyl 3-mercaptopropionate, m-Carborane-1-thiol, m-Carborane-9-thiol, Copper(I) 1-butanethiolate, 4-Cyano-1-butanethiol, S-(4-Cyanobutyl)thioacetate, 1-Decanethiol, 3-(Dimethoxymethylsilyl)-1-propanethiol, 1-Dodecanethiol, tert-Dodecylmercaptan, 2,2′-(Ethylenedioxy)diethanethiol, 2-Ethylhexanethiol, 6-(Ferrocenyl)hexanethiol, Gold Surface Cleaning solution, 1-Heptanethiol, 1,16-Hexadecanedithiol, 1-Hexadecanethiol, Hexa(ethylene glycol)mono-11-(acetylthio)undecyl ether, 1,6-Hexanedithiol, 1-Hexanethiol, 4-Mercapto-1-butanol, 3-Mercapto-2-butanol, mixture of isomers, 12-Mercaptododecanoic acid, 16-Mercaptohexadecanoic acid, 16-Mercaptohexadecanoic acid, 6-Mercaptohexanoic acid, 6-Mercapto-1-hexanol, 4-(6-Mercaptohexyloxy)benzyl alcohol, 3-Mercapto-N-nonylpropionamide, 8-Mercaptooctanoic acid, 15-Mercaptopentadecanoic acid, 1-Mercapto-2-propanol, 3-Mercapto-1-propanol, 3-Mercaptopropionic acid, Mercaptosuccinic acid, 11-Mercaptoundecanoic acid, 11-Mercaptoundecanoic acid, 11-Mercapto-1-undecanol, 11-Mercapto-1-undecanol, (1-Mercaptoundec-11-yl)hexa(ethylene glycol), 11-Mercaptoundecylphosphoric acid, (1-Mercaptoundec-11-yl)tetra(ethylene glycol), 11-Mercaptoundecyl trifluoroacetate, [1-(Methylcarbonylthio)undec-11-yl]tetra(ethylene glycol), Methyl 3-mercaptopropionate, NanoTether BPA-HH, NanoThinks™ 8, NanoThinks™ 18, NanoThinks™ ACID11, NanoThinks™ ACID16, NanoThinks™ ALCO11, NanoThinks™ THIO8, 3,3,4,4,5,5,6,6,6-Nonafluoro-1-hexanethiol purum, 1,9-Nonanedithiol, 1-Nonanethiol, 1-Nonanethiol, 1-Octadecanethiol 9, 1,8-Octanedithiol, 1-Octanethiol, 1-Pentadecanethiol, 1-Pentanethiol, 1H,1H,2H,2H-Perfluorodecanethiol, 1-Propanethiol 99%, 1-Tetradecanethiolpurum, Triethylene glycol mono-11-mercaptoundecyl ether, 1,11-Undecanedithiol and 1-Undecanethiol, or a combination thereof.

The thickness of the thiol self assembled monolayer (SAM) in this study may be about 0.1 nm to about 10 nm, about 0.2 nm to about 9.5 nm, about 0.3 nm to about 9 nm, about 0.4 nm to about 8.5 nm, about 0.5 nm to about 8 nm, about 0.6 nm to about 7.5 nm, about 0.7 nm to about 7 nm, about 0.8 nm to about 6.5 nm, about 0.9 nm to about 6 nm, about 1 nm to about 5 nm, about 1.1 nm to about 4.5 nm, about 1.2 nm to about 4 nm, about 1.3 nm to about 3.5 nm, about 1.4 nm to about 3 nm, about 1.5 nm to about 2.5 nm or about 1.5 nm to about 2 nm. Advantageously, the thickness of the SAM is about 1.5 to about 2 nm.

Sensor design using surface molecular imprinting technology involves coating a support surface, such as a gold substrate for example, with a polymer and embedding template molecules within the polymer layer. Although gold is preferred, other metals such as but not limited to silver, copper, palladium, palladium and mercury and any alloy thereof are also contemplated in the present invention. The high affinity for alkanethiols for the surface of noble and coinage metals makes it possible to generate well-defined organic surfaces with useful and highly alterable chemical functionalities displayed at the chemical surfaces.

Polymer and template molecules may be co adsorbed on a support (e.g., an electrode) surface by soaking the support surface in a suspension containing template molecules and polymer monomers. Advantageously, alkanethiol molecules with hydroxyl end groups and target biological molecules or microbes are co-absorbed on a gold substrate, wherein the thiol molecules chemically attach to the gold surface via sulfer-metal bonds and self-assemble into a stable crystalline monolayer wherein the target molecules (template) are physically incorporated into the thiol monolayer.

Support surfaces may include electrodes, optic fibers, polymer films, metal foil, semiconductors, quartz, glass, and ceramics. After being co-adsorbed on the surface, further polymerization in the presence of the template molecules may occur.

Template molecules may be removed from the polymer layer to provide size-, geometry-, and functionality-specific cavities for target molecules in solution. Since the template molecules are only physically adsorbed onto the support surface, they may be removed by solvent extraction, aging, heat treatment or neutral pH buffer (e.g., phosphate buffered saline), for example. Advantageously, the adsorbed biomolecules are removed from the surface, leaving behind cavities which are complementary in size, shape and chemistry with the template molecules

Without the template molecules embedded in the polymer layer, the electrode is coated with a polymer that contains cavities of specific size, geometry, and functionality according to the template molecule with which they were formed. Mainly, target molecules with size, geometry, and functionality complementary to the cavities may fit in the polymer's cavities and interact with the electrode surface. Thus, the template molecules used during imprinting are the same types of molecules that the sensor is created to detect.

The electro-chemical detection system may employ potentiometry, a technique that identifies specific analytes in solution by measuring the potential of reactions of interest in which those analytes are involved. The surface molecularly imprinted sensor and a reference electrode may be immersed in a solution containing target molecules and other competing molecules. The electrodes are coupled to a potentiometer that measures the potential of reactions of interest occurring in the solution.

In an advantageous embodiment, a two electrode system is used: the Ag/AgCl (saturated KCl) reference electrode and the template/thiol modified sensor or the control as the working electrode. The potential between the working electrode and the reference electrode is measured with a potentiometer (Orion 920). The recorded sensing signal is the potential change of the working electrode after guest molecule addition as compared to that before addition (ΔE=E−E₀, where E is the potential of sensing electrode against reference electrode after addition of the guest molecules and E₀ is that before addition.).

In the present invention, the reaction of interest may be hydrogen bonding between the target molecule in solution and the electrode surface. Since thiol SAM has a blocking effect to electron transfer and most biological molecules are charged in aqueous solutions, the contact point of the charged molecules with the electrode surface will induce a change in the potential of the electrode. Therefore, the accumulation of charged molecules in the cavities can be sensed by a potentiometer.

The target biological molecules encompass any desirable molecules to be quantitated. The biological or chemical molecule may be a cancer marker, vascular marker, inflammatory marker, endocrine marker, metabolic marker, or autoimmune marker.

In an advantageous embodiment, the target biological molecule is a cancer marker.

Advantageously, the cancer marker is a

The cancer marker may be a protein, a peptide, an antibody, an antibody fragment, a receptor, a Cluster Designation/Differentiation (CD) marker, a cytokine, a chemokine, a nucleotide, a lipid, a steroid, a neurotransmitter, a lectin, an imprinted polymer, an oncogene, or an oncogene receptor.

In an advantageous embodiment, the cancer marker is an amylase, which may be a marker of colon, gastric, lung, ovarian, pancreatic or thyroid cancer.

Advantageously, the cancer marker is a saliva marker is selected from the group consisting of tissue polypeptide-specific antigen (TPS), Cyfra 21-1,8-Hydroxy-2′-deoxyguanosine (8OHDG), Squamous cell carcinoma (SCC) antigen, CA 19-9, CA 125, a free radical, a nitrate, a nitrite, a nitric oxide, a carbonyl polypeptide, a thiobarbituric acid reactive substance (TBARS), malondialdehyde (MDA), glutathione S-transferase (GST), Superoxide dismutase (SOD), Uric acid (UA), Ferrylmyoglobin, total antioxidant status (TAS), peroxidase, antioxidant capacity (ImAnOx), Metalloproteinase, Benzodiazepine receptor, pH, Heparanase, total protein, amylase, an electrolyte, lactate dehydrogenase (LDH), insulin-like growth factor (IGF), epidermal growth factor (EGF) and albumin.

Cancer markers may be derived from cancers such as, but not limited to, bladder, breast, cervical, colon, colorectal, gastric, lung, oral, ovarian, pancreatic, prostate and thyroid cancer.

A bladder cancer marker may be selected from the group consisting of AMFr, M-344, 19a21 1, and p53.

A breast cancer marker may be selected from the group consisting of a member of the MUC-type mucin family, a member of the epidermal growth factor receptor (EGFR) family, a carcinoembryonic antigen (CEA), a MAGE (melanoma antigen) gene family antigen, a T/Tn antigen, a hormone receptor, a Cluster Designation/Differentiation (CD) antigen, a tumor suppressor gene, a cell cycle regulator, an oncogene, an oncogene receptor, a proliferation marker, an adhesion molecule, a proteinase involved in degradation of extracellular matrix, a malignant transformation related factor, an apoptosis related factor, a human carcinoma antigen, a member of the vascular endothelial growth factor (VEGF) receptor family, glycoprotein antigens, DF3 antigen, 4F2 antigen or MFGM antigen.

An oral cancer marker may be a p-53 responsive gene 2 product, beta A inhibin, human alpha-1 collagen type I, placental protein 11, BENE protein, neuromedin U, flavin containing monooxygenase 2, runt-related transcription factor 1, alpha 2 collagen type I, fibrillin 1, absent in melanoma 1, nonvoltage-gated 1 alpha sodium channel, protein tyrosine kinase 6 or epithelial membrane protein 1.

An ovarian cancer marker may be CA125.

A prostate cancer marker may be a prostate specific antigen, prostate specific membrane antigen, prostate-specific transglutaminase, cytokeratin 15, semenogelin II or thymosin β 15.

In another advantageous embodiment, the target biological molecule may be a bacteria or a virus.

The bacteria may be selected from the group consisting of Rhodospirillaceae, Chromatiaceae, Chlorobiaceae, Myxococcaceae, Archangiaceae, Cystobacteraceae, Polyangiaceae, Cytophagaceae, Beggiatoaceae, Simonsiellaceae, Leucotrichaceae, Achromatiaceae, Pelonemataceae, Spirochaetaceae, Spirillaceae, Pseudomonadaceae, Azotobacteraceae, Rhizobiceae, Methylomonadaceae, Halobacteriaceae, Enterobacteriaceae, Vibrionaceae, Bacteroidaceae, Neisseriaceae, Veillonellaceae, bacterial organisms oxidizing ammonia or nitrite, bacterial organisms metabolizing sulfur and sulfur compounds, bacterial organisms depositing iron or manganese oxides, Siderocapsaceae, Methanobacteriaceae, Aerobic and facultatively anaerobic Micrococcaceae, Streptococcaceae, Anaerobic Peptococcaceae, Bacillaceae, Lactobacillaceae, Coryneform group of bacteria, Propionibacteriaceae, Actinomycetaceae, Mycobacteriaceae, Frankiaceae, Actinoplanaceae, Dermatophilaceae, Nocardiaceae, Streptomycetaceae, Micromonosporaceae, Rickettsiaceae, Bartonellaceae, Anaplasmataceae, Chlamydiaceae, Mycoplasmataceae, Acholeplasmataceae and Bacillus.

The virus may be selected from the group consisting of Enterovirus, Cardiovirus, Rhinovirus, Aphthovirus, Calicivirus, Orbivirus, Reovirus, Rotavirus, Abibirnavirus, Piscibirnavirus, Entomobirnavirus, Alphavirus, Rubivirus, Pestivirus, Flavivirus, Influenzavirus, Pneumovirus, Paramyxovirus, Morbillivirus, Vesiculovirus, Lyssavirus, Coronavirus, Bunyavirus, Arenavirus, Human immunodeficiency virus, Hepatitis A virus, Hepatitis B virus and Hepatitis C virus.

The present invention also encompasses in vitro and in vivo detection of the target biological molecule. In vivo detection of a target biological molecule may be obtained by placing the sensing element on a transducer and miniaturizing the whole assembly, which is then integrated into a smart chip.

The invention will now be further described by way of the following non-limiting examples.

### EXAMPLES

### Example 1

### Surface Molecular Imprinting—A Novel Approach for Biosensing

The basic principals of the surface molecular imprinting (SMI) technique are illustrated in FIG. 1A. Alkanethiol molecules with hydroxyl end groups and target biological molecules or microbes are co-absorbed onto the gold substrate from the solution. Thiol molecules chemically attached to the gold surface through the sulfur-metal bond and self-assembled into stable crystalline monolayer [Ulman A., Chem. Rev. 1996, 96, 1533], while the target molecules (template) are physically incorporated among the thiol monolayer. Upon repetitive rinsing with water and phosphate buffered saline (PBS) solution, the adsorbed biomolecules are removed from the surface, leaving behind cavities which are complementary in size, shape and chemistry only with the template molecules. Hence, the sensing electrode is expected to have higher affinity to the template molecules than to other guest molecules. The thickness of the 11-mercapto-1-undecanol (thiol) SAM in this study is around 1.5-2 nm [Bain C D., Troughton E B., Tao Y T., Evall J., Whitesides J M., and Nuzzoj R G., J. Am., Chem. Soc., 1989, 111, 321]. Although thicker SAM can be obtained with thiol with longer chain, crystallinity will be decreasing as a result, which reduce the shape memory effect of the SAM, leading to a lower recognition ability. For template molecules much larger than the SAM thickness, only a small portion of the molecule is in contact with the SAM and the electrode surface, in which the shape and size complementarity is remembered by the large number of directional hydrogen bonding, as illustrated in FIG. 1B. Since thiol SAM has blocking effect to electron transfer [Chen H., Heng C. K., Puiu P. D., Zhou X. D., Lee A. C., Lim T. M., Tan S. N., Anal. Chem. Acta 2005, 554, 52], and most of biological molecules are charged in aqueous solutions, the contact point of the charged molecules with the electrode surface will induce a change in the potential of the electrode, and hence the accumulation of the charged individuals in the cavities can be sensed by potentiometer [Janata J., J. Am. Chem. Soc. 1975, 97, 2914].

Carcinoembryonic antigen (CEA) is one of the mostly widely used tumor markers for monitoring colorectal cancer [Michael J. Duffy, Clinical Chemistry. 2001; 47:624-630]. CEA is a large glycoprotein, associated with colon cancer cells, but also found in embryonic tissue. It has a complex structure with carbohydrate side chains whose molecular weight ranges between 180,000 to 200,000 Da and having a radius of gyration R_{g} of 8.0 nm. [Boehm M K., Mayans M O., Thornton J D., Begent R H J., Keep P A., Perkins S J., J. Molecl. Bio. 1996, 259, 718]. The normal concentration of CEA for adults is less than 2.5 ng/ml for non-smokers and less than 5.0 ng/ml for smokers. Benign disease only causes a small increase of CEA in the serum value to no higher than 10 ng/ml. In patients with appropriate symptoms, a five folds increase in the CEA concentration (>5 times the upper limit of normal) is suggests the presence of colorectal cancer. Higher levels, in excess of 20 ng/mL, are usually associated with cancers that have spread [Michael J. Duffy, Clinical Chemistry. 2001; 47:624-630]. Currently the CEA concentrations are generally measured by immunoassay methods, in which the specific antibody-antigen interaction is tracked by either radioactive or fluorescent labeling. SMI can be used to detect CEA as well. The advantages are that the precision is comparable and that this technique avoids the usage of radioactive materials.

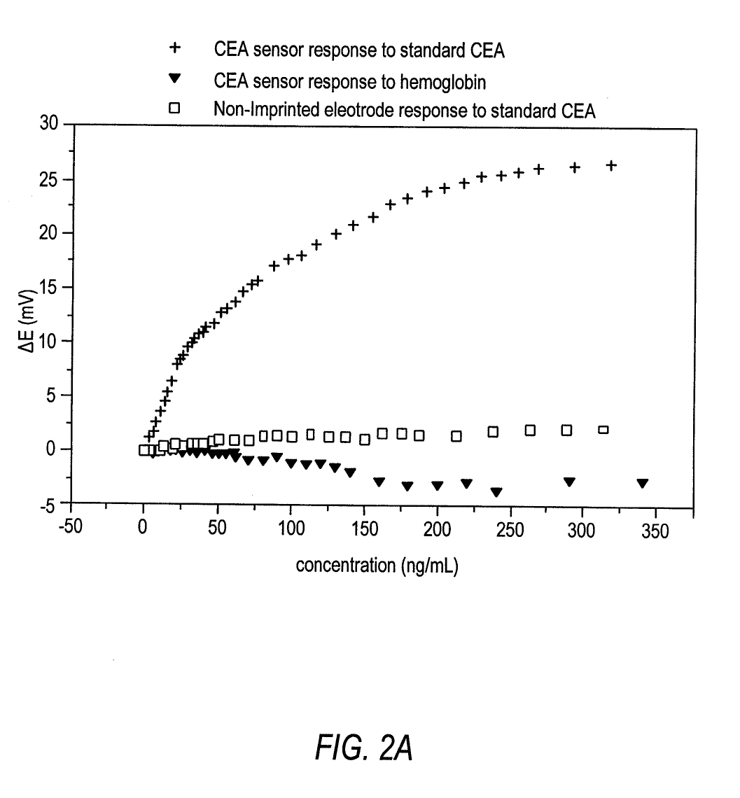

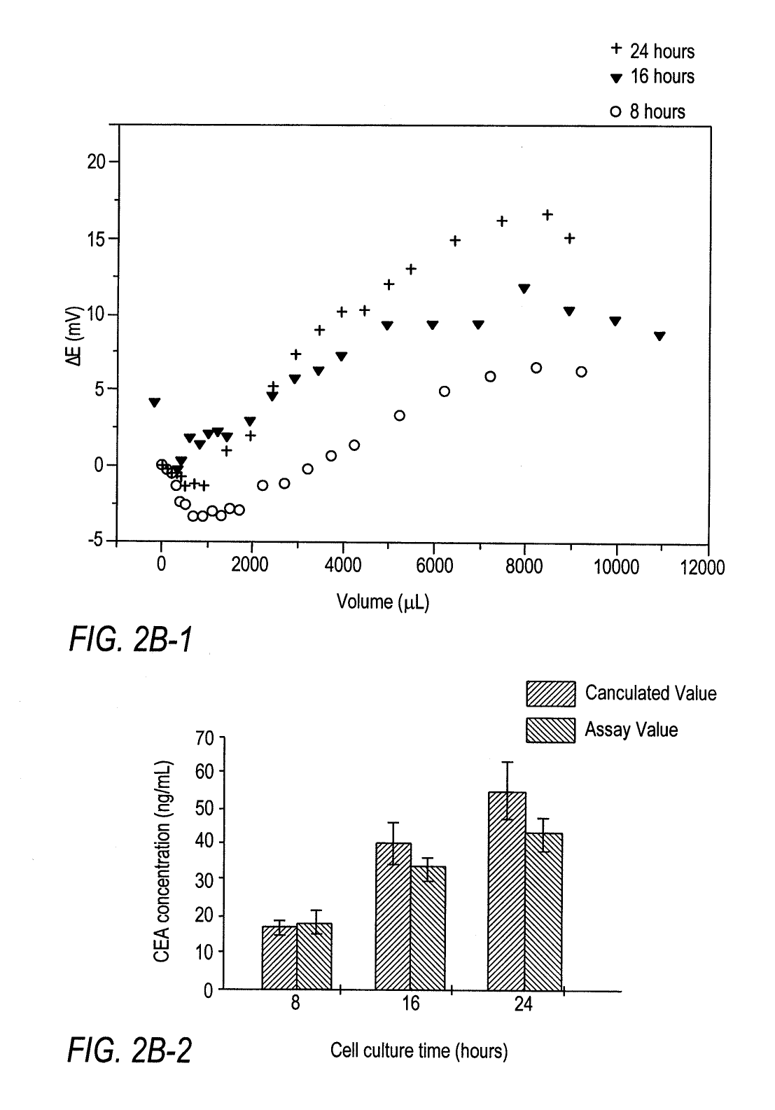

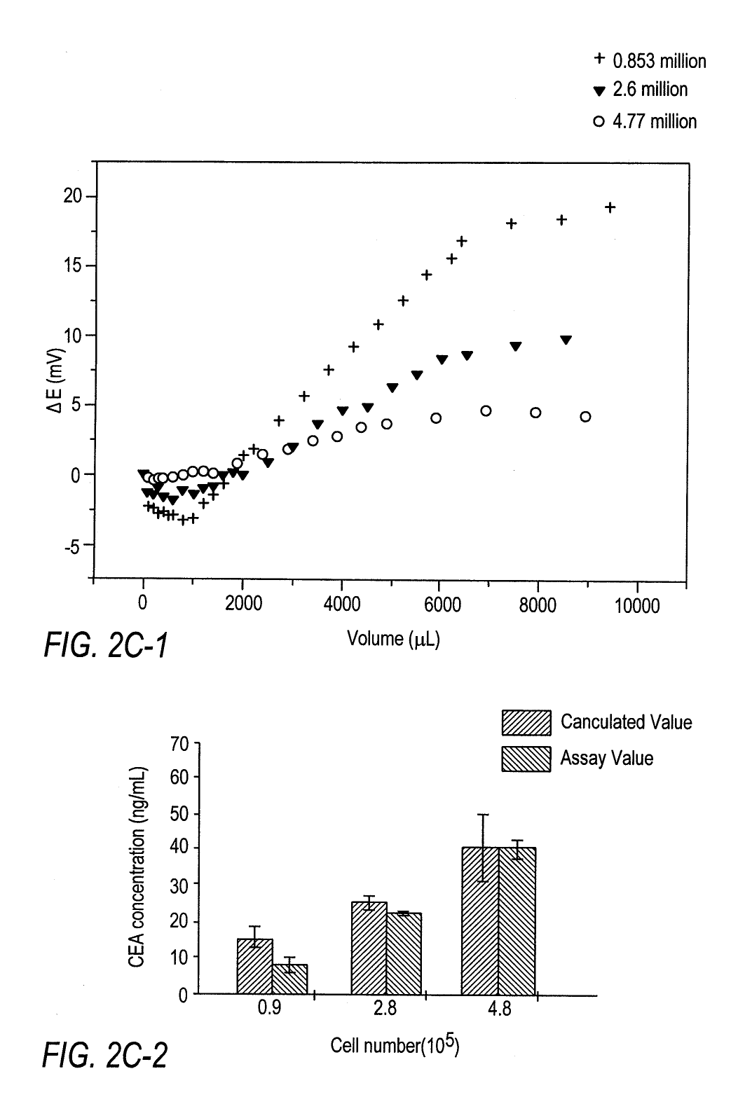

The SMI sensor was fabricated using 0.83 μM solution of CEA molecules and its sensitivity was tested using LoVo colorectal cancer cells which are known to secret a significant amount of CEA while dividing and growing [Drewinko B., Romsdahl M. M., Yang L. Y., Ahearn M. J., Trujillo J. M., Cancer Res. 1976, 36, 467]. FIG. 2A shows the response of the sensor as a function of CEA concentration in Ham's F12K medium along with the sensor response as a function of the hemoglobin concentration, as well as the non-imprinted electrode response to the CEA. From the figure it can be seen that no response is obtained when placed in a solution of hemoglobin proteins or in a solution of CEA when the sensor was not imprinted. On the other hand, for the imprinted electrode the inventors observe a rapid increase in the potential for CEA concentrations below ˜165 ng/mL, followed by a more gradual increase at higher concentrations. This change in potential reflects an accumulation of the surface charge induced by the adsorption of the target molecules, which reaches a stable value due to the saturation of the acceptor sites. The potential increases nearly linearly with CEA concentration in the solution at low concentration range. FIG. 2B shows the sensing electrode response as a function of volume of the medium where LoVo cells were cultured for different periods of time. Here too, the potential difference increases rapidly at low volumes and saturates at higher volumes. From the figure it can be seen that the slope as well as the saturation value increases with incubation time. Using the known CEA marker curve in FIG. 2A as a calibration of the sensor, the concentration of CEA in the original cell medium can be estimated as a function of incubation time. This value is plotted in the inset as purple column where it is seen that the CEA concentration increases monotonically with incubation time. This is consistent with the fact that for the cells cultured for a longer time, the CEA concentration in the medium is higher. To verify the precision of the technique, the CEA concentration in the cell medium was measured by the enzyme immunoassay method, and plotted for comparison. For the 8 hour cells, the concentrations of the CEA tested by the two method differ as little as 1 ng/mL; for the 24 hour cells, the largest difference is 12 ng/mL. Similarly, FIG. 2C shows the potential response of the sensor to the medium of LoVo cells in different numbers, cultured for similar period of time. The CEA concentration in the medium was calculated according to the curve in FIG. 2A and plotted as the insert along with the assay value, from which it will be seen that the concentrations measured by the two methods agree with each other fairly well for the 2.6 million and 4.8 million cells, with difference of less than 2 ng/mL; while for the 0.9 millions cell medium, a slightly bigger difference, 7.5 ng/mL, was reached by the two methods. The results demonstrate that for the medium containing a larger number of cells the inventors find a higher concentration of CEA. Hence molecular imprinting can provide a quantitative determination of the CEA concentration with a precision comparable to immunoassay techniques. Furthermore, since the technique is more robust namely, the recognition element and transducer are integrated, the sensor can be implanted in vivo and the signals transmitted remotely to a detector.

In order to further probe the specificity and sensitivity of the SMI detector to large molecule biomarkers, the inventors also tested its ability to detect another tumor marker, cathepsin D (Cath-D). Cath-D is induced by oestrogen cancer cells where its concentration is correlated with a high risk of metastasis [Spyratos, F., Maundelonde, T., Brouillet, J. P. et al. (1989). Lancet, ii, 8672, 1115-1118, Suzumori N., Ozaki Y., Ogasawara M., Suzumori K., Molecl. Human Reproduction, 2001, 7, 459]. Hence Cath-D can be used as an independent prognostic factor for metastasis of breast cancer since metastatic breast cancer cell lines secrete higher levels of pro-cath-D than do normal mammary cells [Garcia M., Platet N., Liaudet E., Laurent V., Derocq D., Brouillet J. P., Rochefort H., Stem Cells, 1996, 14, 642]. Recently, Fukuda et al have also demonstrated that elevated Cath-D levels were a reliable predictor of short survival rates in glioma patients and that the degree of aggressiveness of the tumor could be gauged by quantitative measurements of the Cath-D serum levels. [Fukuda M. E., Iwadate Y., Machida T., Hiwasa T., Nimura Y., Nagai Y., Takiguchi M., Tanzawa H., Yamaura A., and Seki N., Cancer Res. 2005; 65, 5190]. Hence a detector capable of accurately measuring low levels of Cath-D in vivo can prove to be a very useful predictor of early stages of cancer metastasis.

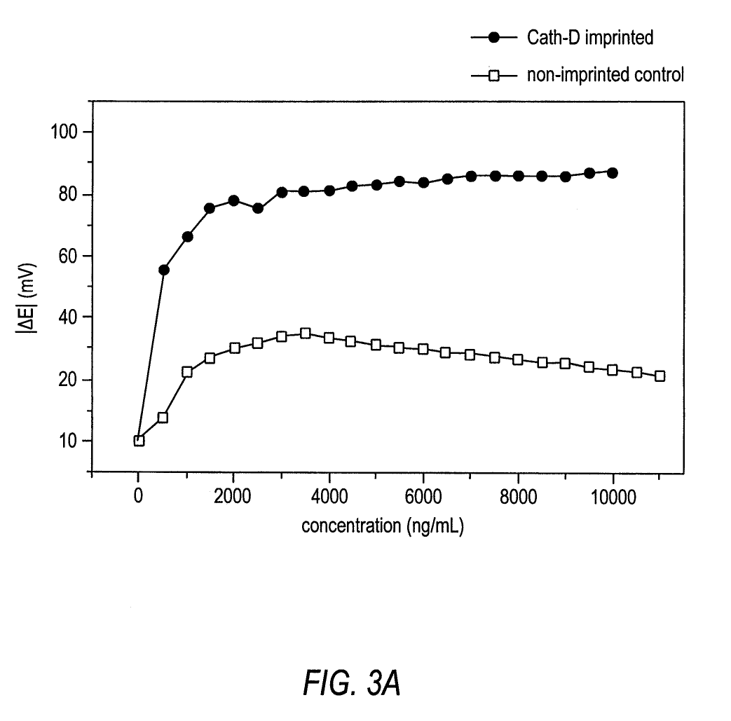

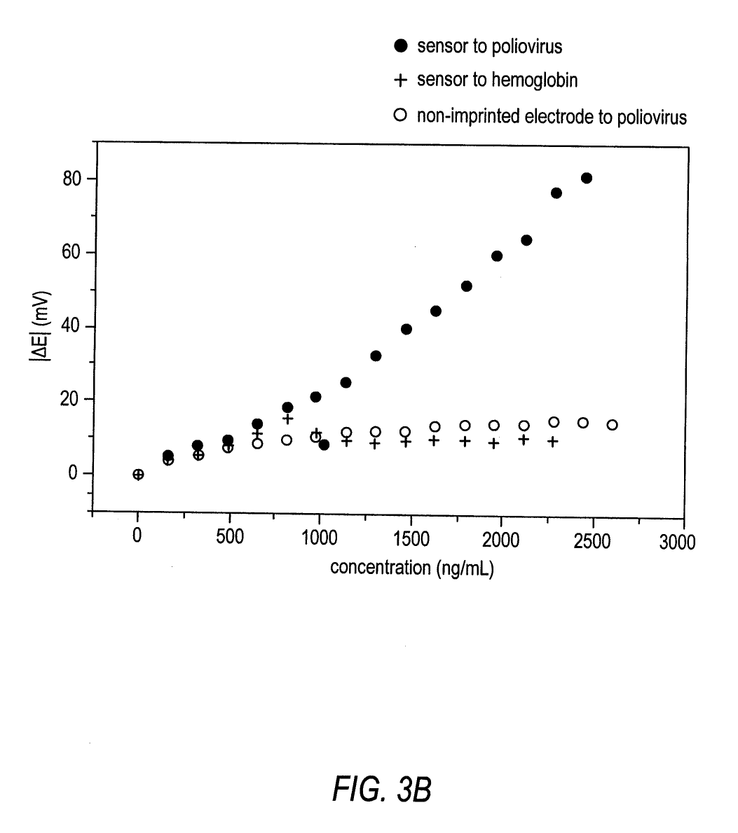

The electrochemical response of the Cath-D templated electrode together with the response of a non-imprinted control electrode to the addition of Cath-D molecules is plotted in FIG. 3A. From the figure it is recognized that the detector is also highly sensitive to nanogram levels of Cath-D, illustrating the versatility of the technique for detecting different types of cancer markers.

To determine if the technique is sensitive to even larger and more complex constructs, the inventors tried to detect poliovirus, which is a positive strand RNA virus within a protein capsid. The capsid surface has a corrugated topography consisting of 60 copies of each of the four different types of proteins [Hogle M J., Chow M., Filman D J., Science, 1985, 229, 1358]. The molecular weight of the viral assembly is ˜8.2 million Da with a diameter of 20-30 nm.

Molecular imprinting was used to template the sensing electrode within a dilute poliovirus/thiol blend solution. The potential response of the electrode to the poliovirus is plotted in FIG. 3B, where it is seen that despite the large size, the electrode is still very sensitive to the presence of the poliovirus. A large, monotonic change of the potential with virus concentration is detected on the imprinted electrode, while the non-imprinted control electrode, (circles) only shows a slight potential change at low concentrations, which can be caused by random capture of the target individuals by the defects or loose points at the SAM, and it saturates very quickly at higher concentrations. The trend suggests that the imprinted electrode maintains its affinity to the polioviruses, despite the fact that the height of the capturing sites is much smaller than the capsid diameter. Since the capsid of the virus consists of proteins, the question arises whether any other proteins, such as those found in the blood plasma, could cause a similar response. The inventors therefore tested the response of a sensor imprinted with poliovirus, but placed in a solution of hemoglobin, a common blood plasma protein. The potential curve is plotted as diamons in FIG. 3B, where it can be seen that the response is similar in magnitude to that of the non-imprinted control electrode. Hence the sensor can detect a specific antigen without interference from other plasma proteins that are present at the same time.

An essential question then arises, namely, if the molecular imprinting is simply a morphological lock and key mechanism, why can the sensor detect molecules whose diameters can be more than ten times larger than the imprinting surface? A possible explanation is suggested in FIG. 1B. In contrast to the assumptions of reference [Zhou Y., Yu B., Shiu E., Levon K., Anal. Chem. 2004, 76, 2689], the templating cavity need not sense the entire templating molecule, if another variable is introduced, namely the orientation of surface functional groups. In order to template with biological molecules, hydroxyl terminated thiol molecules were used which could be self assembled from aqueous solution. An additional benefit of using these molecules is their ability to form the hydrogen bonds with the hydrophilic groups on the protein surface. These interactions are sufficient to attract the target molecules from solution, but not as strong as covalent bonds, which would inhibit their ability to be removed in the templating process. As shown in FIG. 1B, when the template virus enters a complementary cavity, a large number of hydrogen bonds are formed with the capsid surface, which reflect the highly structured order of the surface proteins. Removing the virus, then leaves behind a templated cavity in the crystalline self assembled monolayer, which not only maintains the morphological shape of the molecule, but also the orientation of the surface proteins. Since this orientation is at once specific and local, recognition of the capsid surface could be achieved even if only a small fraction of the capsid presents at the templated electrodes [Shi H., Tsai W. B., Garrison M. D., Ferrari S., Ratner B. D., Nature 1999, 398, 593]. Therefore, molecular imprinting can be far more versatile than originally thought, since the recognition ability depends on chemical complementarity, as well as size and shape.

In conclusion, the use of water compatible hydroxyl terminated thiol chains to produce the SAM extends the technique of surface molecular imprinting to hydrophilic molecules and introduces chemical orientation as another parameter in the template formation. Consequently, the technique can now be used for sensing complex biological molecules such as cancer markers and viruses, which are much larger than the SAM template. The technique was applied to the detection of CEA molecules in solution and produced by living cells in culture. Calibration of the method indicated that quantities as small as 2.5 ng/mL could be detected. Hence the production of the CEA as a function of cell number and incubation time could be tracked. The detector was also used successfully to probe nanogram quantities of Cath-D levels which were previously only detected by immunoassay method and which are accurate indicators of breast cancer and glioma metastatis. Finally the detector was also shown to successfully detect nanogram quantities of poliovirus without interference from other blood proteins in human serum. Since the sensor and transducer are on one element, and can be miniaturized, this detection method can in principal be implanted in vivo, post surgically, for early detection of recurring cancers.

Materials include 11-mercapto-1-undecanol (thiol), lyophilized powder of single chain cathepsin D (cathD) (M_{w}=45,000) from bovine spleen, buffered aqueous solution (≧95%) of carcinoembryonic antigen (CEA) from human fluids (M_{w}=200,000), hemoglobin (M_{w}=64,500) from bovine blood were purchased from Sigma-Aldrich; polio virus (M_{w}=8.2×10⁶) was provided by Dr. Stephen Mueller. All of the above materials are stored in refrigerator at a temperature below 0° C. when not used.

Gold (500 Å) coated silicon substrates were cleaned with ethanol and deionized water and dried with nitrogen gas. Since the proteins or viruses dissolve in aqueous solvent, while the hydroxyl alkanethiol dissolves in organic solvent, blending solvents were used. Proteins or virus (template) were dissolved de-ionized water and thiol was dissolved in acetic acid, respectively, then the two solutions were mixed to make the concentration of thiol to be 10⁻⁴M. To avoid aggregation of the template molecules, low concentration was chosen: 0.244 nM for poliovirus, 0.833 nM for carcinoembryonic, 0.44 μM for Cath-D, which represent the range from 1.47×10¹¹ to 2.67×10¹⁴ molecules/mL. The blend solution was shaken before kept still for 1 hour to allow complete mixing. The gold coated electrode was immersed into the solution at room temperature for at least two hours. Then it was rinsed with de-ionized water for 5 minutes. For comparison, an electrode modified with pure thiol SAM was made without the templates which was used as the control electrode.

The LoVo cells were incubated in the Ham's F12 medium with 2 mM L-glutamin adjusted to contain 1.5 g/L sodium bicarbonate (90%) and fetal bovine serum (10%) at 37° C. for a specific amount of time.

The experiments for electrochemical measurement were made in 8 mL of phosphate buffered saline (PBS: pH=7.15) in a 10 mL working volume electrochemical cell equipped with a magnetic stirrer. Two electrode system was used: the Ag/AgCl (saturated KCl) reference electrode and the template/thiol modified sensor or the control as the working electrode. The potential between the working electrode and the reference electrode was measured with a potentiometer (Orion 920). The recorded sensing signal is the potential change of the working electrode after guest molecule addition as compared to that before addition (ΔE=E−E₀, where E is the potential of sensing electrode against reference electrode after addition of the guest molecules and E₀ is that before addition.).

### Example 2

### Sensing of the Amylase

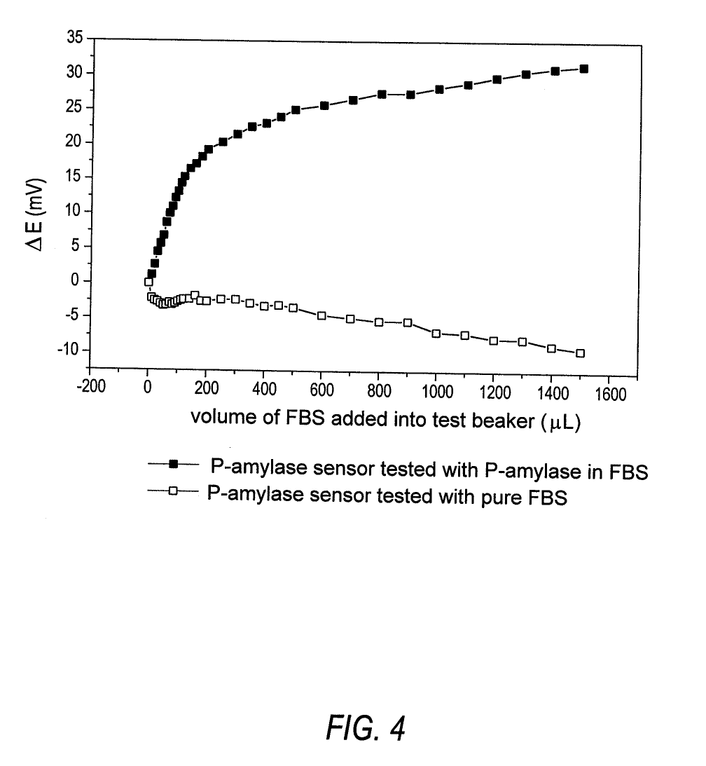

The sensor was fabricated through imprinting with the α-amylase from human pancreas, and the sensitivity was tested in pure FBS as a function of added volume of the FBS containing the same kind of amylase, where a significant potential response was observed. The final concentration of the amylase in the test solution corresponded to 300 ng/mL. As shown in FIG. 4, the sensor showed very slight response to the addition of pure FBS into phosphate buffered saline. Since the pure FBS contained numerous kinds of other proteins, the results again confirmed that the sensor had good selectivity and could recognize the target protein in multi-component protein systems.

### Example 3

### Poliovirus Sensor Test

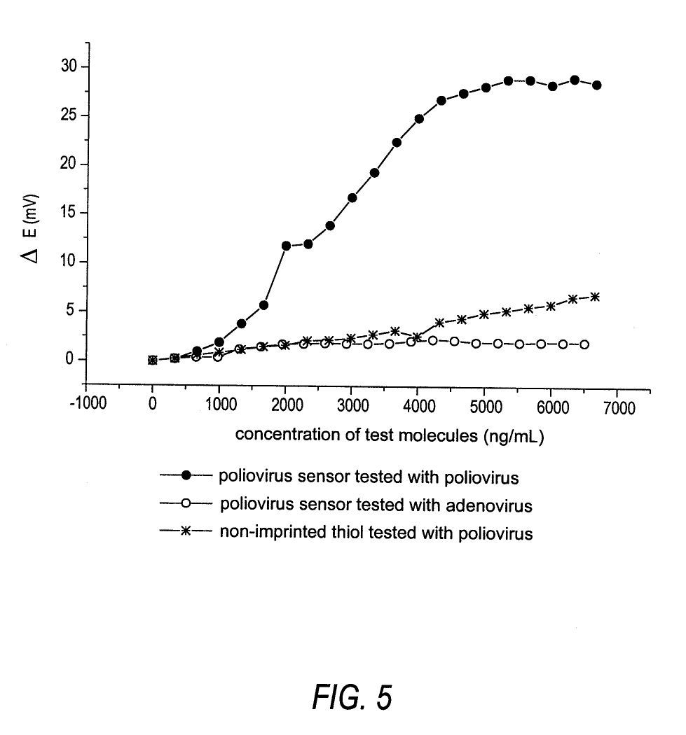

Sensor fabrication included poliovirus (5.32 μg/mL), thiol (20 μg/mL) co-dissolved in DI water, acetic acid, NaOH with a pH=7.00 and a substrate of Au coated silicon. The results are presented in FIG. 5.

The sensor showed a much bigger response to the presence of poliovirus as compared to adenovirus. The non-imprinted electrode is insensitive to poliovirus.

Having thus described in detail preferred embodiments of the present invention, it is to be understood that the invention is not to be limited to particular details set forth in the above description as many apparent variations thereof are possible without departing from the spirit or scope of the present invention.

What is claimed is:

1. A surface-molecularly imprinted sensor for detecting target biological molecules, the sensor comprising:
a) an electrode; and
b) hydroxyl functionalized thio hydrocarbon chains directly assembled on the electrode, wherein the polymer monolayer or film is imprinted with cavities for detecting the target biological molecules.

2. The surface-molecularly imprinted sensor of claim 1 wherein the cavities are complementary to the size, geometry, and functionality of the target biological molecules.

3. The surface-molecularly imprinted sensor of claim 1 wherein the cavities are made by template molecules.

4. The surface-molecularly imprinted sensor of claim 3 wherein the template molecules are target biological molecules.

5. The surface-molecularly imprinted sensor of claims 1 wherein the cavities imprinted in the hydroxyl functionalized thio hydrocarbon chains are specific to carcinoembryonic antigen (CEA), cathepsin-D, poliovirus or amylase.

6. The surface-molecularly imprinted sensor of claim 1 wherein the electrode is a gold-plated electrode.

7. A method for detecting target biological molecules using a surface-molecularly imprinted sensor, the method comprising:
a) providing a solution containing target biological molecules;
b) providing the sensor of claim 1;
c) choosing a detection method; and
d) recognizing target biological molecules based on the detection method's output.

8. The method of claim 7 wherein the detection method is potentiometry and wherein the detection method's output is the potential response of the solution.

9. The method of claim 7 wherein the sensor is implanted in vivo and the detection method's output is transmitted remotely to a detector.
